(19) United States
(12) Patent Application Publication
(10) Pub. No.: US 2016/0153997 A1
(43) Pub. Date: Jun. 2, 2016

(54) OLIGOPEPTIDE SPECIFIC TO COLORECTAL CANCER CELL AND APPLICATION THEREOF

(71) Applicant: National Tsing Hua University, Hsinchu (TW)

(72) Inventors: Gwo-Bin LEE, Hsinchu (TW); Yu-Jui CHE, Hsinchu (TW); Chih-Hung WANG, Hsinchu (TW); Lien-Yu HUNG, Hsinchu (TW)

(21) Appl. No.: 14/661,482

(22) Filed: Mar. 18, 2015

(30) Foreign Application Priority Data
Nov. 27, 2014 (TW) 103141180

(51) Int. Cl. G01N 33/574; C07K 7/08; C07K 7/06

(52) U.S. Cl. CPC G01N 33/57419; C07K 7/06; C07K 7/08

(57) ABSTRACT

The present invention provides an oligopeptide specific to colorectal cancer cells, which are selected from a group of peptide sequences consisting of Seq ID: NO. 1 to Seq ID: NO. 3. The oligopeptide specific to colorectal cancer cells of the present invention may be applied for colorectal cancer detection method having non-invasive, fast and convenient properties

### BACKGROUND OF THE INVENTION

1. Field of the Invention

The present invention relates to a novel oligopeptide sequence and application thereof, particularly to an oligopeptide sequence specific to colorectal cancer cell and application thereof.

2. Description of the Prior Art

Cancer is one of the leading causes of death in humans. Colorectal cancer (CRC) is the most frequently diagnosed cancer causing about 700,000 deaths every year. The earlier the cancer is diagnosed, a significantly increase in the five-year survival rate of the patients is observed. For example, patients diagnosed with stage I CRC have a five-year survival rate higher than 90%. The number drops to less than 10% at stage IV reflecting the importance of early diagnose of CRC.

Traditional methods for CRC diagnosis commonly involved invasive approaches such as digital rectal examination, proctoscopy, flexible sigmoidoscopy, and colofibroscopy. These endoscopy-based methods are generally accurate tests offering advantages such as direct observation of polyps and therefore are wildly used in hospitals. Like other invasive diagnosis methods, these approaches possess a higher risk and can result in discomfort.

Fecal occult blood test (FOBT) is a cheap and simple to perform method, although the false-positive result is generally high. Furthermore, serological tests using carcinoembryonic antigen (CEA) and carbohydrate antigen 19-9 (CA19-9) as biomarkers for CRC diagnosis have also been performed. However, these markers are not specific enough for CRC early detection since patients with pancreatic cancer and lung cancer also show an increase of CEA and CA 19-9 values.

The advancement of cancer therapeutic technology has greatly improved the survival rates of patients with CRC, although recurrence of the cancer is still common. It is recognized now that a small fraction of cancer cells, named cancer stem cells (CSC), show distinct biological features from other cells in the cancer population. Cancer stem cells possess the ability of self-renewal, the capability of developing multiple cell lineages, and the potential of extensive proliferation. Cancer stem cells also display high drug resistance and are therefore difficult to eradicate. If therapies can be targeted against CSCs such that the tumor may lose its ability of growing and maintaining, then it may eventually lead to a complete cure. Cancer stem cells have been identified in CRC, and the cells are known to contribute to metastasis in the patients after receiving chemotherapy. In order to detect or isolate colorectal cancer stem cells (CR-CSCs), certain cell surface molecules including CD44, CD133 (Prominin-1), and EpCAM have been used as biomarkers of CR-CSCs.

However, these molecules are also present in other types of CSCs and do not have sufficient specificity for CR-CSC detection. Therefore, the development of a technology to efficiently identify novel specific biomarkers for CR-CSC and CRC cells detection will contribute greatly in diagnosis and treatment of CRC.

### SUMMARY OF THE INVENTION

The present invention is directed to providing a novel oligopeptide sequence, which is specific to colorectal cancer cell and may be applied in colorectal cancer detection and provide a detection method having non-invasive, fast and convenient properties.

According to an embodiment, an oligopeptide specific to colorectal cancer cells comprises a peptide sequence selected from the group consisting of Seq ID: NO. 1 to Seq ID: NO. 3.

According to another embodiment of the present invention, a method for colorectal cancer detection comprises connecting an oligopeptide specific to colorectal cancer cells with magnetic beads, and then contacting to a test sample for binding reaction; and calculating a binding ratio between the oligopeptide and the test sample.

According to yet another embodiment, the present invention provides a polynucleotide which encodes the said peptide sequence as above.

Other advantages of the present invention will become apparent from the following descriptions taken in conjunction with the accompanying drawings wherein certain embodiments of the present invention are set forth by way of illustration and examples.

### BRIEF DESCRIPTION OF THE DRAWINGS

The foregoing aspects and many of the accompanying advantages of this invention will become more readily appreciated as the same becomes better understood by reference to the following detailed descriptions, when taken in conjunction with the accompanying drawings, wherein:

FIG. 1 is a flowchart diagram illustrating for screening oligopeptides specific to colorectal cancer cells according to one embodiment of the present invention.

### DESCRIPTION OF THE PREFERRED EMBODIMENT

The present invention is further detailed with following preferred embodiments in accompany with drawings. It should be noted that the following experimental data of various disclosed embodiments is used for ease of explanation the technical features of the present invention and not intended to limit to the aspects which may be implemented.

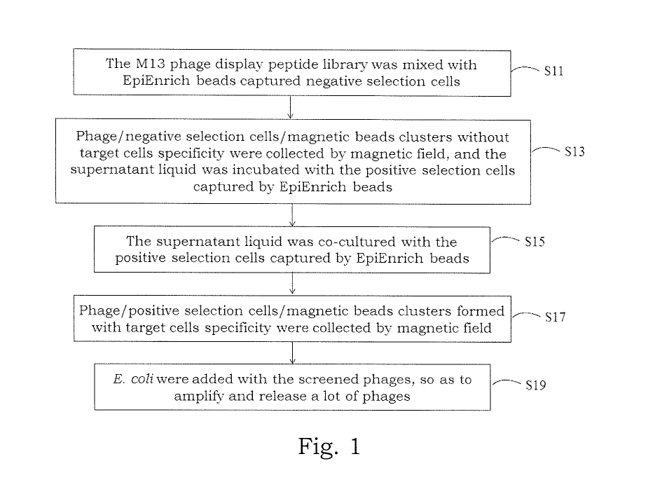

The present invention provides oligopeptide sequences that are capable of specifically binding to colorectal cancer cells and are obtained by following screening steps illustrated in FIG. 1. M13 phage display peptide library and Epithelial Enrich immunomagnetic beads (EpiEnrich beads) were used for selection of cell-surface specific cancer marker of the colorectal cancer cells. In step S11, the M13 phage display peptide library was mixed with EpiEnrich beads captured negative selection cells. Herein, since the present embodiment is intended to select the oligopeptide that specifically binds to colorectal cancer cells, the negative selection cells may use any cell line but the colorectal cancer cells. Then, as illustrated in step S13, phage/negative selection cells/magnetic beads clusters without target cells (that is, colorectal cancer cells) specificity were collected by magnetic field, and the supernatant liquid was obtained. After that, the supernatant liquid was incubated with the positive selection cells captured by EpiEnrich beads, as shown in step S15. Herein, in an embodiment, the positive selection cells may be colorectal cancer cell line (HCT-8). Alternatively, in another embodiment, the cells used for positive selection may be colorectal cancer stem cell (CR-CSC) that are isolated by flow cytometry and enriched by suspension culture. Finally, as illustrated in step S17, phage/positive selection cells/magnetic beads clusters formed with target cells specificity were collected by magnetic field, and unbound phages were washed to complete the primary screening step.

In step S19, Escherichia coli (E. coli) ER2738 were added with the phages selected from the primary screening step, so as to amplify and release a lot of phages, which are used as the phage display library in the secondary screening step. Then, the following steps are screened from a plurality of cycles, preferably, 6 cycles. The screened phages from the above method are collected for nucleotide sequencing and confirming of oligopeptide sequence.

According to the above-described embodiments of the present invention, the peptide sequence having binding specificity and affinity to HCT-8 or CR-CSC are screened, as listed in Seq ID: NO. 1 to Seq ID: NO. 3, and polynucleotide sequences encode the said peptide sequence are listed in Seq ID: NO. 4 to Seq ID: NO. 6.

Capturing Ability Analysis

In one embodiment, the peptide sequences of Seq ID: NO. 1 to Seq ID: NO. 3 are respectively bonded to the beads and mixed with different cell lines , respectively, including colorectal cancer cells (HCT-8), colorectal cancer stem cells (CR-CSC), human hepatoma cells (HepG2), human in situ pancreatic cancer cells (BxPC-3), human lung adenocarcinoma cells (A549) and human breast cancer cells (MCF7). Wherein, the beads having peptide sequences are mixed with each cell line at room temperature for 25 rpm 30 minutes, and the cell number of each cell line is 2×10⁵. After washing for several times, the number of cells captured with magnetic beads having the above peptide sequences are calculated and the results are shown as Table 1 below.

TABLE 1

| | Seq ID: NO. 1 | Seq ID: NO. 2 | Seq ID: NO. 3 |
|---|---|---|---|
| | Capturing | Capturing | Capturing |
| Cell line | ability (%) | ability (%) | ability (%) |
| colorectal cancer | 43.40 ± 7.23 | 45.16 ± 7.12 | 9.42 ± 2.67 |
| cell (HCT-8) | | | |
| colorectal cancer | 22.97 ± 4.29 | 20.85 ± 0.61 | 49.79 ± 5.34 |
| stem cell (CR-CSC) | | | |
| human hepatoma | 23.60 ± 3.50 | 23.02 ± 2.77 | 17.87 ± 6.18 |
| cell (HepG2) | | | |
| human in situ | 3.41 ± 1.14 | 3.13 ± 0.62 | 3.58 ± 1.18 |
| pancreatic cancer | | | |
| cell (BxPC-3) | | | |
| human lung | 28.51 ± 7.09 | 34.29 ± 5.22 | 16.77 ± 6.49 |
| adenocarcinoma | | | |
| cell (A549) | | | |
| human breast | 12.84 ± 7.67 | 20.45 ± 4.04 | 10.68 ± 5.08 |
| cancer cell (MCF7) | | | |

The capture rate of HCT-8 by Seq ID: NO. 1 and Seq ID: NO. 2 are 43.30% and 45.16%, respectively. The capture rate of CR-CSC by Seq ID: NO. 3 is 49.79%. Therefore, the peptide sequences of the present invention provide higher recognition rate to colorectal cancer cell and colorectal cancer stem cell, than that to other tumor cells.

In summary, the oligopeptides of the present invention having the peptide sequences Seq ID: NO. 1 to Seq ID: NO. 3 are provided with higher specificity and affinity to colorectal cancer cell and colorectal cancer stem cell, respectively and may contribute to early detection of colorectal cancer and more convenient and faster methods for detecting cancer.

While the invention can be subject to various modifications and alternative forms, a specific example thereof has been shown in the drawings and is herein described in detail. It should be understood, however, that the invention is not to be limited to the particular form disclosed, but on the contrary, the invention is to cover all modifications, equivalents, and alternatives falling within the spirit and scope of the appended claims.

What is claimed is:

1. An oligopeptide specific to colorectal cancer cells, comprising a peptide sequence selected from the group consisting of Seq ID: NO. 1 to Seq ID: NO. 3.

2. The oligopeptide specific to colorectal cancer cells according to claim 1, wherein the colorectal cancer cells comprise colorectal cancer stem cells.

3. The oligopeptide specific to colorectal cancer cells according to claim 1, wherein the oligopeptide is artificial.

4. A method for colorectal cancer detection, comprising:
contacting an oligopeptide specific to colorectal cancer cells to a test sample for binding reaction, wherein the oligopeptide comprises a peptide sequence selected from the group consisting of Seq ID: NO. 1 to Seq ID: NO. 3; and
calculating a binding ratio between the oligopeptide and the test sample.

5. The method for colorectal cancer detection according to claim 4, wherein the colorectal cancer cells comprise colorectal cancer stem cells.

6. The method for colorectal cancer detection according to claim 4, wherein the oligopeptide binds on the magnetic beads.

7. The method for colorectal cancer detection according to claim 4, wherein the test sample comprises cells or tissues.

8. The method for colorectal cancer detection according to claim 4, further comprising:
determining if the test sample comprises colorectal cancer cells based on the binding ratio.

9. A polynucleotide, wherein the sequence of the poly nucleotide encodes the peptide sequence as an oligopeptide specific to colorectal cancer cells, which comprises a peptide sequence selected from the group consisting of Seq ID: NO. 1 to Seq ID: NO. 3.

10. The polynucleotide as claim 9, wherein the colorectal cancer cells comprise colorectal cancer stem cells.

11. The polynucleotide as claim 9, wherein the oligopeptide is artificial.

12. The polynucleotide as claim 9, the sequence of the polynucleotide is selected from the group consisting of Seq ID: NO. 4 to Seq ID: NO. 6.


SEQUENCE LISTING (6 sequences)

SEQ ID NO: 1 - protein, 9 aa, Artificial Sequence
  synthesized
AELAKLPLF

SEQ ID NO: 2 - protein, 10 aa, Artificial Sequence
  synthesized
GAISFDHVHP

SEQ ID NO: 3 - protein, 12 aa, Artificial Sequence
  synthesized
MNAKWPIENLMN

SEQ ID NO: 4 - DNA, 36 bp, Artificial Sequence
  synthesized
gcggagcttgctaagttgccgctgttttagatgcgc

SEQ ID NO: 5 - DNA, 36 bp, Artificial Sequence
  synthesized
ggtgctatttcttttgatcatgtgcatccttagagg

SEQ ID NO: 6 - DNA, 36 bp, Artificial Sequence
  synthesized
atgaatgctaagtggccgattgagaatttgatgaat